(19) United States
(12) Patent Application Publication
(10) Pub. No.: US 2004/0171640 A1
(43) Pub. Date: Sep. 2, 2004

(54) Substituted 1-phenethylpiperidine compounds used as inter alia analgesics

(76) Inventors: Bernd Sundermann, Aachen (DE); Lambert Hoenen, Aachen (DE); Helmut Buschmann, Esplugues de Llobregat (ES); Babette-Yvonne Koegel, Langerwehe-Hamich (DE); Elmar Friderichs, Stolberg (DE)

Correspondence Address: Clarence A. Green, 425 Post Road, Fairfield, CT, 06824, US

(21) Appl. No.: 10/751,584

(22) Filed: Jan. 5, 2004

Related U.S. Application Data
(63) Continuation of application No. PCT/EP02/07379, filed on Jul. 3, 2002.

(30) Foreign Application Priority Data
Jul. 5, 2001 (DE) 101 32 746.3

(51) Int. Cl. A61K 31/454; C 07D 4 1/02; A61K 31/445

(52) U.S. Cl. 514/317; 514/326; 546/207; 546/229

(57) ABSTRACT

The invention relates to substituted 1-Phenethylpiperidine compounds, a method for the production thereof, medicaments containing said compounds and the use of said compounds in the production of medicaments.

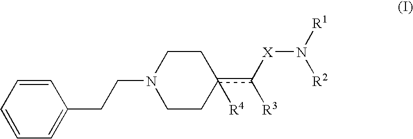

The present invention relates to substituted 1-phenethylpiperidine compounds, a process for the production thereof, pharmaceutical preparations containing these compounds and the use of these compounds for the production of pharmaceutical preparations.

Pain is one of the basic clinical symptoms. There is a worldwide need for effective pain treatments. The urgency of the requirement for effective therapeutic methods for providing tailored and targeted treatment of chronic and non-chronic pain, this being taken to mean pain treatment which is effective and satisfactory from the patient's standpoint, is evident from the large number of scientific papers relating to applied analgesia and to basic nociception research which have appeared in recent times.

Conventional opioids, such as for example morphine, are effective in the treatment of severe to very severe pain. However, they produce accompanying symptoms which include respiratory depression, vomiting, sedation, constipation and development of tolerance. Research is being carried out worldwide into other pain-relieving agents.

The object of the present invention was accordingly to provide new active ingredients which are particularly suitable as pharmaceutical active ingredients in pharmaceutical preparations.

These active ingredients are intended to be particularly suitable for the combatting of pain, for the treatment of migraine, diarrhoea, urinary incontinence, pruritus, inflammatory reactions, allergic reactions, dependency on alcohol and/or drugs and/or medicines, abuse of alcohol and/or drugs and/or medicines, inflammation or for local anaesthesia.

According to the invention, this object is achieved by the provision of substituted 1-phenethylpiperidine compounds of general formula I below, which exhibit a pronounced analgesic effect and which are also suitable in particular for the treatment of migraine, diarrhoea, urinary incontinence, pruritus, inflammatory reactions, allergic reactions, dependency on alcohol and/or drugs and/or medicines, abuse of alcohol and/or drugs and/or medicines, inflammation or for local anaesthesia.

The present invention accordingly provides substituted 1-phenethylpiperidine compounds of the general formula I

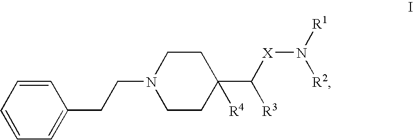

in which

X denotes a methylene (CH₂) or carbonyl (C═O) group, preferably a methylene (CH₂) group,

R¹ denotes an optionally at least mono-substituted aryl or heteroaryl residue, preferably an optionally at least mono-substituted aryl residue,

R² denotes H, COR⁵, SO₂R⁵, an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or an optionally at least mono-substituted aryl or heteroaryl residue attached via a C₁₋₃ alkylene group, preferably H, COR⁵, SO₂R⁵ or a C₁₋₆ alkyl residue, particularly preferably H or COR⁵,

R³ and R⁴ each separately denote H or together denote a bond, preferably each separately denote H,

R⁵ denotes an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or an optionally at least mono-substituted aryl or heteroaryl residue attached via a C₁₋₃ alkylene group, preferably a C₁₋₆ alkyl residue or an optionally at least mono-substituted aryl residue,

as a free base or a corresponding physiologically acceptable salt and corresponding racemates, enantiomers and diastereomers.

The aliphatic residues may be mono- or polysubstituted. If these residues comprise more than one substituent, these may be identical or different and attached both to the same and to different atoms of the aliphatic residue. The aliphatic residues may preferably be substituted with a halogen residue and/or a hydroxyl group, particularly preferably with F and/or Cl.

Saturated aliphatic residues may preferably be selected from the group consisting of optionally at least mono-substituted methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl and 1-methylpentyl. Substituted aliphatic residues may particularly preferably be CHF₂ or CF₃.

Unsaturated aliphatic residues may preferably selected from the group consisting of vinyl (ethenyl), allyl (2-propenyl) and 1-propynyl.

The cycloaliphatic residues may be mono- or polysubstituted. If the cycloaliphatic residues comprise more than one substituent, these may be identical or different and be attached both to the same and to different atoms of the cycloaliphatic residue. The cycloaliphatic residues may preferably be substituted with a halogen residue and/or a hydroxyl group, preferably with fluorine and/or chlorine.

The cycloaliphatic residues may preferably be selected from the group consisting of optionally at least mono-substituted cyclopropyl, 2-methylcyclopropyl, cyclopropylmethyl, cyclobutyl, cyclopentyl, cyclopentylmethyl, cyclohexyl, cycloheptyl and cyclooctyl.

The term aryl residue also includes for the purposes of the present invention those aromatic hydrocarbon residues, which are fused with a saturated or at least partially unsaturated hydrocarbon ring system. An optionally at least mono-substituted phenyl or naphthyl residue is preferred as aryl residue.

If the aryl residue comprises more than one substituent, these may be identical or different. Preferably, these substituents are selected from the group consisting of OR⁶, halogen, preferably F and/or Cl, CF₃, CN, NO₂, NR⁷R⁸, C₁₋₆ alkyl, C₁₋₆ alkoxy, C₃₋₈ cycloalkoxy and unsubstituted phenyl or phenyl at least mono-substituted with OR⁶, halogen, preferably F and/or Cl, CF₃, CN, NO₂, NR⁷R⁸, C₁₋₆ alkyl, C₁₋₆ alkoxy or C₃₋₈ cycloalkoxy and unsubstituted naphthyl or naphthyl at least mono-substituted with OR⁶, halogen, preferably F and/or Cl, CF₃, CN, NO₂, NR⁷R⁸, C₁₋₆ alkyl, C₁₋₆ alkoxy or C₃₋₈ cycloalkoxy, wherein

R⁶ denotes H, a C₁₋₁₀ alkyl residue, preferably a C₁₋₆ alkyl residue, an unsubstituted aryl or heteroaryl residue or denotes an unsubstituted aryl or heteroaryl residue attached via a C₁₋₃ alkylene group,

R⁷ and R⁸, identical or different, denote H, a C₁₋₁₀ alkyl residue, preferably a C₁₋₆ alkyl residue, an unsubstituted aryl or heteroaryl residue or denote an unsubstituted aryl or heteroaryl residue attached via a C₁₋₃ alkylene group, or the residues R⁷ and R⁸ together mean the group —CH₂CH₂OCH₂CH₂—, —CH₂CH₂NR⁹CH₂CH₂—, or —(CH₂)₃-6, wherein

the residue R⁹ denotes H, a C₁₋₁₀ alkyl, preferably a C₁₋₆ alkyl, an unsubstituted aryl or heteroaryl residue or denotes an aryl or heteroaryl residue attached via a C₁₋₃ alkylene group.

For the purposes of the present invention, a heteroaryl residue is understood to mean also those heteroaromatic, preferably 5- or 6-membered hydrocarbon residues, which are fused with a saturated or partially unsaturated hydrocarbon ring system. Preferably, the heteroaryl residues contain one or more heteroatoms selected from the group consisting of nitrogen, oxygen and sulfur.

Preferred heteroaryl residues are selected from the group consisting of unsubstituted or at least mono-substituted furan, benzofuran, thiophene, benzothiophene, pyrrole, pyridine, pyrimidine, quinoline, isoquinoline, phthalazine and quinazoline.

If the heteroaryl residue comprises more than one substituent, these may be identical or different. Preferably, these substituents are selected from the group consisting of OR⁶, halogen, preferably F and/or Cl, CF₃, CN, NO₂, NR⁷R⁸, C₁₋₆ alkyl, C₁₋₆ alkoxy, C₃₋₈ cycloalkoxy, unsubstituted phenyl and unsubstituted naphthyl,

wherein the residues R⁶, R⁷ and R⁸ have the above-stated meaning. The following substituted 1-phenethylpiperidine compounds and the corresponding physiologically acceptable salts thereof, preferably the hydrochlorides thereof, are very particularly preferred:

2-(1-Phenethylpiperidin-4-yl)-N-phenylacetamide,

[2-(1-Phenethylpiperidin-4-yl-)ethyl]phenylamine,

2-(1-Phenethylpiperidin-4-ylidene)-N-phenyl-acetamide,

N-(2-Methoxyphenyl)-2-(1-phenethylpiperidin-4-yl)-acetamide,

N-(4-Methoxyphenyl)-2-(1-phenethylpiperidin-4-yl)-acetamide,

2-(1-Phenethylpiperidin-4-yl)-N-(2-trifluormethoxyphenyl)acetamide,

(4-Methoxyphenyl)-[2-(1-phenethylpiperidin-4-yl)ethyl]amine,

2-[2-(1-Phenethylpiperidin-4-yl)ethylamino]phenol,

N-(3-Methoxyphenyl)-2-(1-phenethylpiperidin-4-yl)acetamide,

N-(3-Chloro-4-methoxyphenyl)-2-(1-phenethylpiperidin-4-yl)acetamide,

N-(4-Chloro-2-fluorophenyl)-2-(1-phenethylpiperidin-4-yl)acetamide,

2-(1-Phenethylpiperidin-4-yl)-N-(3-trifluoromethylphenyl)acetamide,

[2-(1-Phenethylpiperidin-4-yl)ethyl]-(3-trifluoromethylphenyl)amine,

(3-Methoxyphenyl)-[2-(1-phenethylpiperidin-4-yl)ethyl]amine,

4-[2-(1-Phenethylpiperidin-4-yl)ethylamino]phenol,

(4-Chloro-2-fluorophenyl)-[2-(1-phenethylpiperidin-4-yl)ethyl]amine,

3-[2-(1-Phenethylpiperidin-4-yl)ethylamino]phenol,

N-(3-Chloro-4-methoxyphenyl)-N-[2-(1-phenethylpiperidin-4-yl)ethyl]acetamide,

N-(3-Chloro-4-methoxyphenyl)-N-[2-(1-phenethylpiperidin-4-yl)ethyl]propionamide,

N-(3-Chloro-4-methoxyphenyl)-N-[2-(1-phenethylpiperidin-4-yl)ethyl]benzamide,

N-[2-(1-Phenethylpiperidin-4-yl)ethyl]-N-(3-trifluoromethylphenyl)acetamide,

N-[2-(1-Phenethylpiperidin-4-yl)ethyl]-N-phenylacetamide,

N-[2-(1-Phenethylpiperidin-4-yl)ethyl]-N-phenylbenzamide,

(4-Methylpyridin-2-yl)-[2-(1-phenethyl-piperidin-4-yl)-ethyl]amine and

(4,6-Dimethyl-pyridin-2-yl)-[2-(1-phenethylpiperidin-4-ylidene)-ethyl]amine.

The present invention further provides a process for the production of substituted 1-phenethylpiperidine compounds of the above-stated general formula I, according to which

(a) 1-phenethylpiperidin-4-one of the formula II

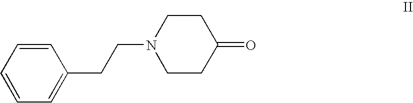

is reacted with triethyl phosphonoacetate in solution to yield (1-phenethylpiperidin-4-ylidene)-ethyl acetate of the formula III

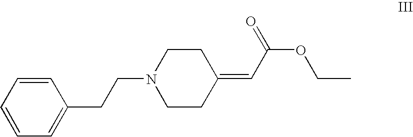

and this is optionally purified in accordance with conventional methods and/or optionally isolated in accordance with conventional methods,

(b) optionally the (1-phenethylpiperidin-4-ylidene)-ethyl acetate of the formula III is converted in accordance with conventional methods into a compound of the general formula IV,

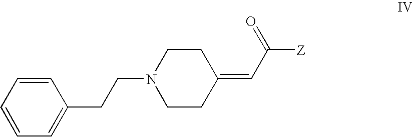

in which Z denotes a group which activates the carbonyl carbon atom for reaction with an amine, the compound of the general formula IV thus obtained is optionally purified in accordance with conventional methods and/or optionally isolated in accordance with conventional methods,

(c) optionally at least one of the compounds of the formula III or IV in solution is reduced to yield a corresponding compound of the general formula III′

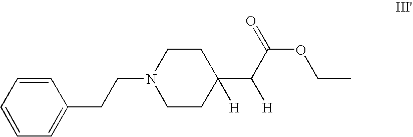

or to yield a corresponding compound of the general formula IV′

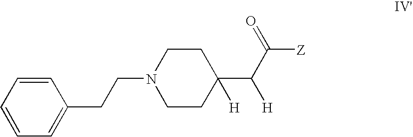

and the corresponding compound is optionally purified in each case in accordance with conventional methods and/or optionally isolated in each case in accordance with conventional methods,

(d) at least one compound of the formula III, III′, IV and IV′ in solution is reacted with a primary or secondary amine of the general formula V,

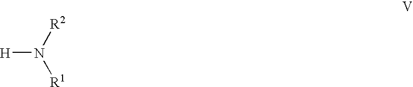

in which R¹ and R² have the meaning according to the above-stated general formula I, to yield at least one compound of the general formula Id

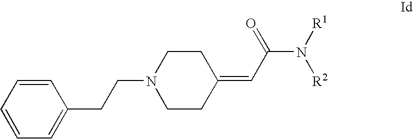

and/or at least one compound of the general formula Id′

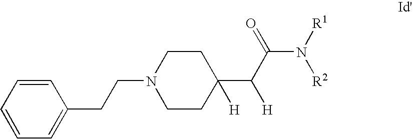

and this is optionally purified in each case in accordance with conventional methods and/or optionally isolated in each case in accordance with conventional methods,

(e) optionally at least one of the compounds of the general formula Id and/or Id′ is converted by reduction in solution into at least one compound of the general formula Ie

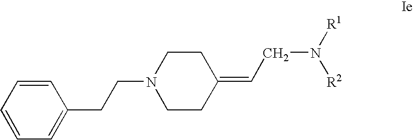

and/or at least one compound of the general formula Ie′

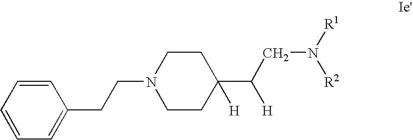

in which R¹ and R² each have the above-stated meaning, and this is optionally purified in each case in accordance with conventional methods and/or optionally isolated in each case in accordance with conventional methods,

(f) optionally at least one compound of the general formula Ie and/or Ie′, in which the residue R² denotes H, is converted in accordance with conventional methods known to the person skilled in the art into at least one compound of the general formula Ie and/or Ie′, in which the residue R² denotes COR⁵, SO₂R⁵, an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or denotes an optionally at least mono-substituted aryl or heteroaryl residue attached via a C₁₋₃ alkylene group, wherein the residue R⁵ has the above-stated meaning and this is optionally purified in accordance with conventional methods and/or optionally isolated in accordance with conventional methods.

The starting compounds to be used in each case and the required reagents are generally commercially available or may be produced according to conventional methods known to the person skilled in the art.

The solvents and reaction conditions used correspond to the solvents and reaction conditions conventional for these types of reactions. These are known to the person skilled in the art for example from A. P. Gray et al., J. Org. Chem., 26, 1961, pages 3368-3373, P. C. Jain et al., Indian J. Chem. 10, 1972, pages 455-460, T. Weida et al., J. Med. Chem. 39, 1996, pages 380-387, H. Sugimoto et al., J. Med. Chem. 33, 1990, pages 1880-1887, P. Bernard et al., J. Comp. Aided Mol. Desig. 13, 1999, pages 355-371 and the literature cited in each thereof. The corresponding literature descriptions are hereby introduced as a reference and are deemed to be part of the disclosure.

The conversion of the (1-phenethylpiperidin-4-ylidene)-ethyl acetate of the formula III into a compound of the general formula IV, in which the carbonyl carbon atom for reaction is activated with an amine, may proceed in accordance with conventional methods known to the person skilled in the art, such as described for example in M. Bodansky, “The Peptides”, volume 1, 1979, pages 105-196. The corresponding literature description is hereby introduced as a reference and is deemed to be part of the disclosure.

In a preferred embodiment of the process according to the invention the group Z denotes OH, Cl or succinimide.

The reduction of the compounds of the formula III or IV to yield the compounds of the formula III′ or IV′ may proceed in accordance with conventional methods known to the person skilled in the art. In a preferred embodiment of the process according to the invention, the reduction proceeds with hydrogen in the presence of a transition metal catalyst, preferably in the presence of palladium powder, in a suitable solvent. The reduction may be performed at various hydrogen pressures, preferably at a hydrogen pressure of 1 to 200 bar, preferably 1 to 5 bar.

Reaction of the compounds of the general formula III, III′, IV and IV′ with primary or secondary amines of the general formula V may proceed in accordance with conventional methods known to the person skilled in the art. The reaction with a primary or secondary amine of the general formula V preferably proceeds in the presence of n-butyllithium.

The reduction of the compound of the general formula Id or Id′ to yield compounds of the general formula Ie or Ie′ may proceed in accordance with conventional methods known to the person skilled in the art. In a preferred embodiment of the process according to the invention, the reduction proceeds with lithium aluminium hydride in organic solution or with aluminium hydride (alane), which is formed in situ from lithium aluminium hydride and aluminium chloride.

The substituted 1-phenethylpiperidine compounds according to the invention of the general formula I may be isolated by the process according to the invention both in the form of their free base and in the form of a salt. The free base of the respective compound according to the invention of the general formula I may preferably be converted by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid, into the corresponding physiologically acceptable salt.

Conversion of the free base of the respective compound according to the invention of the general formula I into the corresponding hydrochloride may likewise preferably also be obtained by combining the compound according to the invention of the general formula I, dissolved in a suitable organic solvent, such as for example butan-2-one (methyl ethyl ketone), as a free base with trimethylsilyl chloride (TMSCl).

The free base of the respective compound according to the invention of the general formula I may also preferably be converted with the free acid or a salt of a sugar substitute, such as for example saccharin, cyclamate or acesulfame, into the corresponding physiologically acceptable salt.

If the substituted 1-phenethylpiperidine compounds according to the invention of the general formula I comprise a phenol residue, these may be produced by ether cleavage in accordance with conventional methods known to the person skilled in the art from the corresponding methyl ether. The ether cleavage preferably proceeds with protonic or Lewis acids or with diisobutylaluminium hydride. The cleavage of methyl esters may likewise preferably proceed with aluminium hydride (alane), which is preferably formed in situ from lithium aluminium hydride and aluminium chloride.

If the substituted 1-phenethylpiperidine compounds according to the invention of the general formula I are obtained by the production process according to the invention in the form of the racemates thereof or other mixtures of their various enantiomers and/or diastereomers, these may be separated and optionally isolated by conventional processes known to the person skilled in the art. Examples are chromatographic separation processes, in particular liquid chromatography processes at standard pressure or at elevated pressure, preferably MPLC and HPLC methods, and fractional crystallisation processes. Individual enantiomers, e.g. diastereomeric salts formed by means of HPLC on a chiral phase or by means of crystallisation with chiral acids, such as (+)-tartaric acid, (−)-tartaric acid or (+)-10-camphorsulfonic acid, may here in particular be separated from one another.

The substituted 1-phenethylpiperidine compounds according to the invention of the general formula I are toxicologically safe and are therefore suitable as pharmaceutical active ingredients in pharmaceutical preparations.

The present invention therefore also provides pharmaceutical preparations which contain at least one substituted 1-phenethylpiperidine compound of the general formula I according to the invention and optionally physiologically acceptable auxiliary substances.

If the substituted 1-phenethylpiperidine compounds according to the invention of the general formula I or the corresponding physiologically acceptable salts thereof are chiral, they may be present in the pharmaceutical preparation according to the invention in form of the racemates thereof, the pure enantiomers thereof, the pure diastereomers thereof, or in the form of a mixture of at least two of the above-stated stereoisomers. The substituted 1-phenethylpiperidine compounds according to the invention of the general formula I may likewise also be present in the pharmaceutical preparation in the form of mixtures of the enantiomers or diastereomers thereof. These mixtures may comprise the respective stereoisomers in any desired mixing ratio.

The pharmaceutical preparations according to the invention are preferably suitable for the combatting of pain or for the treatment of migraine, diarrhoea, urinary incontinence, pruritus, inflammatory reactions, allergic reactions, dependency on alcohol and/or drugs and/or medicines, abuse of alcohol and/or drugs and/or medicines, inflammation or for local anaesthesia.

The present invention likewise provides the use of at least one substituted 1-phenethylpiperidine compound of the general formula I to produce a pharmaceutical preparation for the combatting of pain, for the treatment of migraine, diarrhoea, urinary incontinence, pruritus, inflammatory reactions, allergic reactions, dependency on alcohol and/or drugs and/or medicines, abuse of alcohol and/or drugs and/or medicines, inflammation or for local anaesthesia.

The pharmaceutical preparations according to the invention may be present as liquid, semisolid or solid dosage forms, for example in the form of solutions for injection, drops, succi, syrups, sprays, suspensions, tablets, patches, capsules, transdermal delivery systems, suppositories, ointments, creams, lotions, gels, emulsions, aerosols or in multiparticulate form, for example in the form of pellets or granules, and also administered as such.

In addition to at least one substituted 1-phenethylpiperidine compound of the general formula I according to the invention, the pharmaceutical preparations according to the invention conventionally contain further physiologically acceptable pharmaceutical auxiliary substances, which are preferably selected from the group consisting of matrix materials, fillers, solvents, diluents, surface-active substances, dyes, preservatives, suspending agents, slip agents, lubricants, aromas and binders.

Selection of the physiologically acceptable auxiliary substances and the quantities thereof which are to be used depends upon whether the pharmaceutical preparation is to be administered orally, subcutaneously, parenterally, intravenously, intraperitoneally, intradermally, intramuscularly, intranasally, buccally, rectally or topically, for example onto infections of the skin, mucous membranes or eyes. Preparations in the form of tablets, coated tablets, capsules, granules, pellets, drops, succi and syrups are preferred for oral administration, while solutions, suspensions, readily reconstitutible dried preparations and sprays are preferred for parenteral, topical and inhalatory administration. Compounds according to the invention of the general formula I in a depot in dissolved form or in a dressing, optionally with the addition of skin penetration promoters, are suitable percutaneous administration preparations. Orally or percutaneously administrable preparations may also release the compounds of the general formula I according to the invention in delayed manner.

Production of the pharmaceutical preparations according to the invention proceeds with the assistance of conventional means, devices, methods and processes known to the person skilled in the art, such as are described for example in “Remington's Pharmaceutical Sciences”, ed. A. R. Gennaro, 17th ed., Mack Publishing Company, Easton, Pa. (1985), in particular in part 8, chapters 76 to 93. The corresponding literature description is hereby introduced as a reference and is deemed to be part of the disclosure.

The quantity of the respective substituted 1-phenethylpiperidine compound of the general formula I according to the invention to be administered to the patient may vary and is for example dependent on the weight or age of the patient and on the mode of administration, the indication and the severity of the complaint. Conventionally, at least one substituted 1-phenethylpiperidine compound of the general formula I according to the invention is administered in a quantity of 0.005 to 500 mg/kg, preferably of 0.05 to 5 mg/kg, of patient body weight.

Pharmacological Investigations:

1.) Analgesic Testing by Writhing Test in Mice

The investigation into analgesic efficacy was performed by phenylquinone-induced writhing in mice (modified after: I. C. Hendershot J. Forsaith, J. Pharmacol. Exp. There. 125, 237-240 (1959)). The corresponding literature description is hereby introduced as a reference and is deemed to be part of the disclosure.

Male NMRI mice weighing from 25 to 30 g were used for this purpose. Groups of 10 animals per substance dose received, 10 minutes after intravenous administration of the compounds tested, 0.3 ml/mouse of a 0.02% aqueous solution of phenylquinone (phenylbenzoquinone, Sigma, Deisenhofen; solution prepared with addition of 5% of ethanol and stored in a water bath at 45° C.) administered intraperitoneally. The animals were placed individually in observation cages. A push button counter was used to record the number of pain-induced stretching movements (writhing reactions=straightening of the torso with stretching of the rear extremities) for 5-20 minutes after phenylquinone administration. The control was provided by animals who received only physiological common salt solution.

The compounds were tested at the standard dosage of 10 mg/kg. Inhibition of the writhing reactions by a substance was calculated according to the following formula:

$$\% Inhibition = 100 - [ \frac{Writhing reaction , treated animals}{Writhing reaction , control} \times 100 ]$$

2.) Analgesic Testing by Tail Flick Test in Mice

The mice were each individually put in a test cage and the base of the tail was exposed to the focused heat flux from an electric lamp (tail flick type 50/08/1.bc, Labtec, Dr. Hess). The lamp intensity was so set that the time from switching on of the lamp until sudden flicking away of the tail (pain latency) in untreated mice amounted to 3 to 5 seconds. Prior to administration of the solutions containing the compound according to the invention or the respective comparison solutions, the mice were pre-tested twice within five minutes and the average value of these measurements was calculated as a pre-test average value.

The solutions of the compound according to the invention of the general formula I and the comparison solutions were then administered intravenously. Pain was measured in each case 10, 20, 40 and 60 minutes after intravenous administration. The analgesic action was determined as an increase in pain latency (% of the maximum possible antinociceptive effect) in accordance with the following formula:

[(T₁−T₀)/(T₂−T₀)]×100

In this formula, the time T₀ is the latency time prior to administration, the time T₁ is the latency time after administration of active ingredient combination and the time T₂ is the maximum exposure period (12 seconds)

The invention is explained below with reference to Examples. These explanations are given merely by way of example and do not restrict the general concept of the invention.

### EXAMPLES

The yields of the example compounds according to the invention were not optimised.

All temperatures are uncorrected.

Silica gel 60 (0.040-0.063 mm) from E. Merck, Darmstadt, was used as the stationary phase for the column chromatography.

The thin-layer chromatographic investigations were performed with pre-coated silica gel 60 F 254 HPTLC plates from E. Merck, Darmstadt.

The mobile solvent mixture ratios for chromatographic investigations are stated by volume/volume

(1-Phenethylpiperidin-4-ylidene)ethyl acetate (ester 1)

50.0 g (246 mmol) 1-phenethylpiperidin-4-one were dissolved in a mixture of 200 ml sodium hydroxide solution (32 wt. %) and 300 ml of toluene at room temperature. With ice bath cooling, 110 g (491 mmol) of triethyl phosphonoacetate were added dropwise, the ice bath was removed and stirring of the reaction mixture thus obtained was continued for a further 30 minutes. Then the reaction mixture was refluxed for 1.5 hours.

The organic phase was separated off, washed with approx. 100 ml of water, dried over sodium sulfate, filtered and evaporated completely in a vacuum at 500 to 20 mbar. The crude product (60 g) thus obtained was purified by column chromatography with ether as mobile solvent (column size: length 50 cm, diameter 8 cm). 52.8 g of (1-phenethylpiperidin-4-ylidene)ethyl acetate were obtained, corresponding to 79% of the theoretically calculated yield.

(1-Phenethylpiperidin-4-ylidene)ethyl acetate (ester 2)

50.0 g (182 mmol) of (1-phenethylpiperidin-4-ylidene)ethyl acetate (ester 1) were dissolved in 480 ml of ethyl acetate and, after the addition of 0.1 g palladium at a hydrogen pressure of 2 bar, were hydrogenated until hydrogen absorption ceased. The reaction mixture thus obtained was filtered and completely evaporated in a vacuum. 49.0 g of (1-phenethylpiperidin-4-ylidene)ethyl acetate were obtained, corresponding to 97% of the theoretically calculated yield.

General Procedure 1

1.1 mol equivalents of the respective primary amine were dissolved in tetrahydrofuran (approx. 2 ml per mmol amine), 2.2 mol equivalent of n-butyllithium solution (1.6 mol/l in hexane) were added dropwise with ice bath cooling and stirring was continued for an hour. Then the reaction solution was cooled by means of a dry ice bath and a solution of the respective ester (1 mol equivalent) in tetrahydrofuran (approx. 0.5 ml per mmol of ester) was added dropwise. Stirring was continued for an hour with dry ice cooling and the solution was heated overnight. After addition of half-saturated ammonium chloride solution (approx. 2.5 ml per mmol ester) were extracted repeatedly with ether, ethyl acetate or dichloromethane, the extracts thus obtained were combined, dried over sodium sulfate, filtered and completely evaporated in a vacuum at a pressure of 500 to 20 mbar.

For purification, the crude product thus obtained was dissolved in 2-butanone (8.5 ml per g of crude product) optionally after washing with hexane (approx. 8 ml per g of crude product), dry methanol was optionally added in order to dissolve the crude product completely, 0.5 mol equivalent of water and 1.1 mol equivalent of chlorotrimethylsilane were added and the mixture was stirred overnight. The hydrochloride thus obtained was filtered out and dried in a high vacuum.

The substituted 1-phenethylpiperidine compounds produced by way of example according to general procedure 1, the ether used in each case, the solvent used for extraction, the type of purification and the yield in % of the theoretically determined yield are stated in Table 1 below:

TABLE 1

| | | | | Purification | | |
|---|---|---|---|---|---|---|
| Example | | Ester | | Hexane | Methanol | Yield in % |
| no. | Compound | used | Extraction | washing | addition | as hydrochloride |
| 1 | 2-(1-Phenethyl-piperidin-4-yl)- | 2 | Ethyl acetate | — | X | 67 |
| | N-phenylacetamide | | | | | |
| 3 | 2-(1-Phenethyl-piperidine-4- | 1 | Diethyl ether | — | — | 77 |
| | ylidene)-N-phenylacetamide | | | | | |
| 4 | N-(2-Methoxyphenyl)-2-(1- | 2 | Ethyl acetate | X | — | 73 |
| | phenethylpiperidin-4-yl)- | | | | | |
| | acetamide | | | | | |
| 5 | N-(4-Methoxyphenyl)-2-(1- | 2 | Dichloromethane | — | X | 76 |
| | phenethyl-piperidin-4-yl)- | | | | | |
| | acetamide | | | | | |
| 6 | 2-(1-Phenethyl-piperidin-4-yl)- | 2 | Ethyl acetate | X | — | 63 |
| | N-(2-trifluoromethoxy- | | | | | |
| | phenyl)acetamide | | | | | |
| 9 | N-(3-Methoxyphenyl)-2-(1- | 2 | Diethyl ether | — | — | 69 |
| | phenethyl-piperidin-4- | | | | | |
| | yl)acetamide | | | | | |
| 10 | N-(3-Chloro-4- | 2 | Dichloromethane | — | — | 69 |
| | methoxyphenyl)-2-(1- | | | | | |
| | phenethylpiperidin-4- | | | | | |
| | yl)acetamide | | | | | |
| 11 | N-(4-Chloro-2-fluorophenyl)- | 2 | Ethyl acetate | X | — | 22 |
| | 2-(1-phenethylpiperidin-4- | | | | | |
| | yl)acetamide | | | | | |
| 13 | [2-(1-Phenethyl-piperidin-4- | 2 | Diethyl ether | — | — | 94 |
| | yl)ethyl]-(3-trifluoromethyl- | | | | | |
| | phenyl)amine | | | | | |

General Procedure 2:

Three mol equivalent of lithium aluminium hydride (2.3 mol/l in tetrahydrofuran) were reacted in tetrahydrofuran (approx. 1.3 ml per mmol of lithium aluminium hydride) with one mol equivalent of aluminium chloride, stirring was continued for an hour and then one mol equivalent of the respective amide, dissolved in tetrahydrofuran (approx. 2 ml per mmol of amide), was added. Stirring was continued overnight at 20 to 25° C. For working up, the batch was made basic by addition of potassium hydroxide solution (3 mol/l) and extraction was performed repeatedly with ether. The combined extracts were dried over sodium sulfate, filtered, evaporated and the corresponding hydrochloride was precipitated according to general procedure 1.

The substituted 1-phenethylpiperidine compounds produced by way of example according to general procedure 2 and the yield in % of the theoretically determined yield are stated in Table 2 below:

TABLE 2

| Example | | Yield of |
|---|---|---|
| no. | Compound | hydrochloride in % |
| 2 | [2-(1-Phenethylpiperidin-4-yl-) | 73 |
| | ethyl]phenylamine | |
| 7 | (4-Methoxyphenyl)-[2-(1- | 97 |
| | phenethylpiperidin-4- | |
| | yl)ethyl]amine | |
| 8^{a} | 2-[2-(1-Phenethylpiperidin-4- | 29 |
| | yl)ethylamino]phenol | |
| 12 | 2-(1-Phenethylpiperidin-4-yl)-N- | 58 |
| | (3-trifluoromethyl- | |
| | phenyl)acetamide | |
| 14 | (3-Methoxyphenyl)-[2-(1- | 73 |
| | phenethylpiperidin-4- | |
| | yl)ethyl]amine | |
| 16 | (4-Chloro-2-fluorophenyl)-[2-(1- | 70 |
| | phenethylpiperidin-4- | |
| | yl)ethyl]amine | |

a: During production of this compound according to the Examples, the methyl ether was simultaneously also cleaved.

General Procedure 3:

To three mol equivalent of diisobutylaluminium hydride (1.5 mol/l in toluene) there was added dropwise with stirring at a temperature of 20 to 25° C. one mol equivalent of the corresponding methyl ether, dissolved in toluene (2 ml per mmol of the methyl ether), and then refluxing was performed overnight. After cooling, ethanol and water (in each case 1 ml per mmol of ethyl ether) were added dropwise with ice bath cooling and stirring in such a way that the temperature did not rise above 20° C. Then the batch was left to rest for approx. 4 hours in the ice bath, before suction filtration through diatomaceous earth. Washing was then performed with toluene, the filtrate evaporated and the corresponding hydrochloride precipitated according to general procedure 1.

The substituted 1-phenethylpiperidine compounds produced by way of example according to general procedure 3 and the yield in % of the theoretically determined yield are stated in Table 3 below:

TABLE 3

| | | Yield of |
|---|---|---|
| Example no. | Compound | hydrochloride in % |
| 15 | 4-[2-(1-Phenethylpiperidin-4- | 42 |
| | yl)ethylamino]phenol | |
| 17 | 3-[2-(1-Phenethylpiperidin-4- | 69 |
| | yl)ethylamino]phenol | |

General Procedure 4:

One equivalent of the respective amine was dissolved in dichloromethane (approx. 5 ml per mmol) and a spatula-tipful (approx. 5 to 20 mg) of 4-dimethylaminopyridine and 1.05 mol equivalent of triethylamine were added. The batch was cooled with an ice/methanol bath, 1.05 mol equivalent of the respective acid chloride were added dropwise and then stirring was continued for two hours with heating at a temperature of 20 to 25° C.

For working up, the batch was made basic with diluted potassium hydroxide solution (approx. 5 ml per mmol; 2-3 mol/l), stirred briefly and then extracted repeatedly with dichloromethane. The combined extracts were dried over magnesium sulfate, filtered, evaporated and the corresponding hydrochloride was precipitated according to general procedure 1.

The substituted 1-phenethylpiperidine compounds produced by way of example according to general procedure 4 and the yield in % of the theoretically determined yield are stated in Table 4 below:

TABLE 4

| | | Yield of |
|---|---|---|
| Example no. | Compound | hydrochloride in % |
| 18 | N-(3-Chloro-4-methoxyphenyl)- | 59 |
| | N-[2-(1-phenethylpiperidin-4- | |
| | yl)ethyl]acetamide | |
| 19 | N-(3-Chloro-4-methoxyphenyl)- | 43 |
| | N-[2-(1-phenethylpiperidin-4- | |
| | yl)ethyl]propionamide | |
| 20 | N-(3-Chloro-4-methoxyphenyl)- | 59 |
| | N-[2-(1-phenethylpiperidin-4- | |
| | yl)ethyl]benzamide | |
| 21 | N-[2-(1-Phenethylpiperidin-4- | 26 |
| | yl)ethyl]-N-(3- | |
| | trifluoromethylphenyl)acetamide | |
| 22 | N-[2-(1-Phenethylpiperidin-4- | 73 |
| | yl)ethyl]-N-phenylacetamide | |
| 23 | N-[2-(1-Phenethylpiperidin-4- | 71 |
| | yl)ethyl]-N- | |
| | phenylpropionamide | |
| 24 | N-[2-(1-Phenethylpiperidin-4- | 65 |
| | yl)ethyl]-N-phenylbenzamide | |

Pharmacological Investigations:

1.) Analgesic Testing by Writhing Test in Mice:

The in-depth investigation into analgesic efficacy was performed using phenylquinone-induced writhing in mice, as described above.

The investigated compounds according to the invention exhibited an analgesic action. The results of selected writhing investigations are summarised in Table 5 below.

2.) Analgesic Testing by Tail Flick Test in Mice:

The in-depth investigation into analgesic efficacy was performed using the tail flick test in mice, as described above.

The investigated compounds according to the invention exhibited an analgesic action. The results of selected investigations are likewise summarised in Table 5 below.

TABLE 5

| Example | Antinociceptive action in % | |
|---|---|---|
| no. | relative to control group^{b} | PAIN MODEL |
| 1 | 56 (0.1) | Tail flick |
| 2 | 100 (10) | Writhing |
| 3 | 69 (10) | Writhing |
| 4 | 100 (10) | Writhing |
| 5 | 100 (10) | Writhing |
| 6 | 99 (10) | Writhing |
| 7 | 100 (10) | Writhing |
| 8 | 100 (10) | Writhing |
| 9 | 56 (1) | Writhing |
| 10 | 100 (10) | Writhing |
| 11 | 100 (10) | Writhing |
| 12 | 21 (1) | Tail flick |
| 13 | 31 (1) | Tail flick |
| 14 | 100 (10) | Writhing |
| 15 | 100 (10) | Writhing |
| 16 | 80 (10) | Writhing |
| | 31 (10) | Tail flick |
| 17 | 100 (10) | Writhing |
| 18 | 100 (10) | Writhing |
| 19 | 100 (10) | Writhing |
| | 35 (1) | Tail flick |
| 20 | 88 (10) | Writhing |
| | 23 (1) | Tail flick |
| 21 | 100 (10) | Writhing |
| | 34 (1) | Tail flick |
| 22 | 24 (1) | Tail flick |
| 23 | 99 (10) | Writhing |
| 24 | 100 (10) | Writhing |
| | 24 (1) | Tail flick |

b: dosage in mg/kg for intravenous administration is in each case in brackets.

The investigated substituted 1-phenethylpiperidine compounds according to the Examples exhibit good analgesic efficacy.

1. Substituted 1-phenethylpiperidine compounds of the general formula I 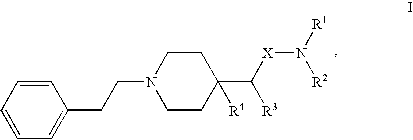
in which
X denotes a methylene (CH₂) or carbonyl (C═O) group,
R¹ denotes an optionally at least mono-substituted aryl or heteroaryl residue,
R² denotes H, COR⁵, SO₂R⁵, an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or an optionally at least mono-substituted aryl or heteroaryl residue attached via a C₁₋₃ alkylene group,
R³ and R⁴ each separately denote H or together denote a bond,
R⁵ denotes an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or an optionally at least mono-substituted aryl or heteroaryl residue attached via a C₁₋₃ alkylene group,
as a free base or a corresponding physiologically acceptable salt and corresponding racemates, enantiomers and diastereomers.

2. Substituted 1-phenethylpiperidine compounds according to claim 1, characterised in that X denotes a methylene (CH₂) group.

3. Substituted 1-phenethylpiperidine compounds according to claim 1 or 2, characterised in that R¹ denotes an optionally at least mono-substituted aryl residue.

4. Substituted 1-phenethylpiperidine compounds according to one of claims 1 to 3, characterised in that R² denotes H, COR⁵, SO₂R⁵ or denotes a C₁₋₆ alkyl residue, preferably denotes H or COR⁵.

5. Substituted 1-phenethylpiperidine compounds according to one of claims 1 to 4, characterised in that the residues R³ and R⁴ each denote H.

6. Substituted 1-phenethylpiperidine compounds according to one of claims 1 to 5, characterised in that the residue R⁵ denotes a C₁₋₆ alkyl residue or denotes an unsubstituted or at least mono-substituted aryl residue.

8. A process for the production of substituted 1-phenethylpiperidine compounds of the general formula I according to one of claims 1 to 7, characterised in that
(a) 1-phenethylpiperidin-4-one of the formula II 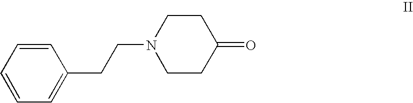
is reacted with triethyl phosphonoacetate in solution to yield (1-phenethylpiperidin-4-ylidene)-ethyl acetate of the formula III 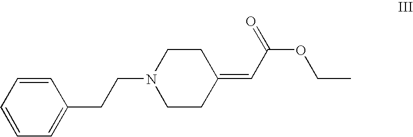
and this is optionally purified in accordance with conventional methods and/or optionally isolated in accordance with conventional methods,
(b) optionally the (1-phenethylpiperidin-4-ylidene)-ethyl acetate of the formula III is converted in accordance with conventional methods into a compound of the general formula IV, 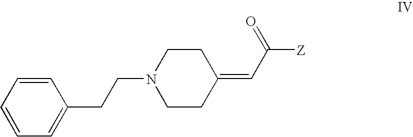
in which Z denotes a group which activates the carbonyl carbon atom for reaction with an amine, the compound of the general formula IV thus obtained is optionally purified in accordance with conventional methods and/or optionally isolated in accordance with conventional methods,
(c) optionally at least one of the compounds of the formula III or IV in solution is reduced to yield a corresponding compound of the general formula III′ 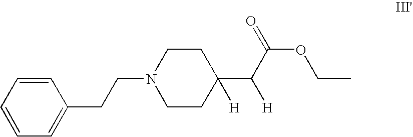
or to yield a corresponding compound of the general formula IV′ 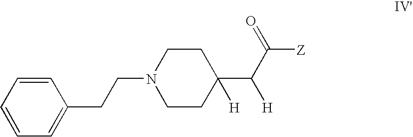
and the corresponding compound is optionally purified in each case in accordance with conventional methods and/or optionally isolated in each case in accordance with conventional methods,
(d) at least one compound of the formula III, III′, IV and IV′ in solution is reacted with a primary or secondary amine of the general formula V, 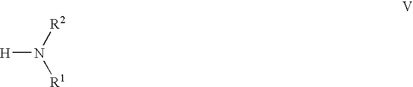
in which R¹ and R² have the meaning according to the above-stated general formula I, to yield at least one compound of the general formula Id 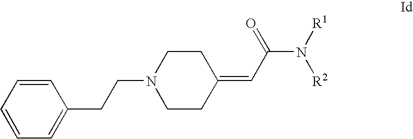
and/or at least one compound of the general formula Id′ 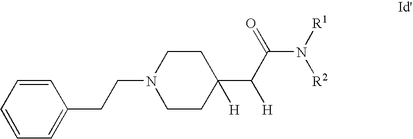
and this is optionally purified in each case in accordance with conventional methods and/or optionally isolated in each case in accordance with conventional methods,
(e) optionally at least one of the compounds of the general formula Id and/or Id′ is converted by reduction in solution into at least one compound of the general formula Ie 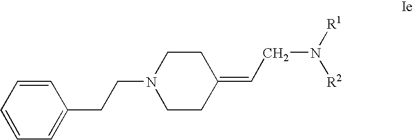
and/or at least one compound of the general formula Ie′ 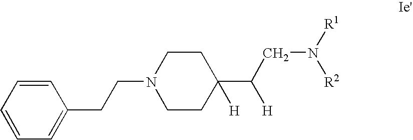
in which R¹ and R² each have the meaning according to claim 1, and this is optionally purified in each case in accordance with conventional methods and/or optionally isolated in each case in accordance with conventional methods,
(f) optionally at least one compound of the general formula Ie and/or Ie′, in which the residue R² denotes H, is converted in accordance with conventional methods known to the person skilled in the art into at least one compound of the general formula Ie and/or Ie′, in which the residue R² denotes COR⁵, SO₂R⁵, an optionally at least mono-substituted, saturated, branched or unbranched aliphatic C₁₋₁₀ residue, an optionally at least mono-substituted, at least mono-unsaturated, branched or unbranched aliphatic C₂₋₁₀ residue, an optionally at least mono-substituted, saturated or at least mono-unsaturated cycloaliphatic C₃₋₈ residue, an optionally at least mono-substituted aryl or heteroaryl residue or denotes an optionally at least mono-substituted aryl or heteroaryl residue attached via a C₁₋₃ alkylene group, wherein the residue R⁵ has the above-stated meaning and this is optionally purified in accordance with conventional methods
and/or optionally isolated in accordance with conventional methods.

9. A process according to claim 8, characterised in that Z denotes OH, Cl or a succinimide residue.

10. A process according to claim 8 or 9, characterised in that the reduction to yield the compounds of formula III′ or IV′ is performed with hydrogen in the presence of a transition metal catalyst, preferably in the presence of palladium powder.

11. A process according to one of claims 8 to 10, characterised in that the reaction with a primary or secondary amine of the general formula V is performed in the presence of n-butyllithium.

12. A process according to one of claims 8 to 11, characterised in that reduction to yield a compound of the general formula Ie or Ie′ proceeds with aluminium hydride (alane) produced in situ from lithium aluminium hydride and aluminium trichloride in an organic solvent.

13. A pharmaceutical preparation containing at least one substituted 1-phenethylpiperidine compound according to one of claims 1 to 7 and optionally physiologically acceptable auxiliary substances.

14. A pharmaceutical preparation according to claim 13 for combatting pain.

15. A pharmaceutical preparation according to claim 13 for the treatment of migraine.

16. A pharmaceutical preparation according to claim 13 for the treatment of diarrhoea.

17. A pharmaceutical preparation according to claim 13 for the treatment of urinary incontinence.

18. A pharmaceutical preparation according to claim 13 for the treatment of pruritus.

19. A pharmaceutical preparation according to claim 13 for the treatment of inflammatory reactions.

20. A pharmaceutical preparation according to claim 13 for the treatment of allergic reactions.

21. A pharmaceutical preparation according to claim 13 for the treatment of the abuse of alcohol and/or drugs and/or medicines.

22. A pharmaceutical preparation according to claim 13 for the treatment of dependency on alcohol and/or drugs and/or medicines.

23. A pharmaceutical preparation according to claim 13 for the treatment of inflammation.

24. A pharmaceutical preparation according to claim 13 for local anaesthesia.

25. Use of at least one substituted 1-phenethylpiperidine compound according to one of claims 1 to 7 to produce a pharmaceutical preparation for the combatting of pain, for the treatment of migraine, diarrhoea, urinary incontinence, pruritus, inflammatory reactions, allergic reactions, dependency on alcohol and/or drugs and/or medicines, abuse of alcohol and/or drugs and/or medicines, inflammation or for local anaesthesia.
